(19) United States
(12) Patent Application Publication
(10) Pub. No.: US 2017/0266370 A1
(43) Pub. Date: Sep. 21, 2017

(54) INJECTION DEVICES WITH TISSUE SWELLING DETECTION

(71) Applicant: Sanofi, Paris (FR)

(72) Inventor: Zdenek Cerman, Frankfurt am Main (DE)

(21) Appl. No.: 15/509,464

(22) PCT Filed: Sep. 11, 2015
(86) PCT No.: PCT/EP2015/070875

§ 371 Date: Mar. 7, 2017

(30) Foreign Application Priority Data
Sep. 15, 2014 (EP) 14306424.4

(51) Int. Cl. A61M 5/142; A61M 5/42; A61M 5/158; A61M 5/168

(52) U.S. Cl. CPC A61M 5/14248; A61M 5/16836; A61M 5/427; A61M 5/158; A61M 2005/14252; A61M 2005/1585; A61M 2005/1588; A61M 2205/3306; A61M 2205/332; A61M 2205/18; A61M 2005/1586; A61M 2205/50; A61M 2205/8206

(57) ABSTRACT

An injection device comprising a housing; a securing arrangement for securing the injection device against an injection site of a patient; a needle insertion mechanism configured to cause insertion of a needle into a patient when the injection device is secured against the injection site of the patient; a medicament delivery arrangement configured to cause injection of a medicament into tissue of the patient at the injection site; and a medicament retention sensor configured to detect retention of medicament in the tissue of the patient at the injection site, wherein the injection device is configured to respond to detection of swelling of the tissue of the patient at the injection site by causing provision of an alert and/or by causing suspension or halting of injection of medicament.

### CROSS-REFERENCE TO RELATED APPLICATIONS

This application is a U.S. national stage application under 35 USC §371 of International Application No. PCT/EP2015/070875, filed on Sep. 11, 2015, which claims priority to European Patent Application No. 14306424.4, filed on Sep. 15, 2014, the entire contents of which are incorporated herein by reference.

### TECHNICAL FIELD

This invention relates to injection devices.

### BACKGROUND

Injection or infusion pumps of the type known as patch pumps for delivering injections of medicament are known in the art. Another type of injection pump that is gaining traction is the bolus injector device.

Some bolus injector devices are intended to be used with relatively large volumes of medicament, typically at least 1 ml and maybe a few ml. Injection of such large volumes of medicament can take some minutes or even hours. Such high capacity bolus injector devices can be called large volume devices (LVDs).

To use a bolus injector device such as an LVD, it is first supported on a suitable injection site of a patient and, once installed, injection is initiated by the patient or another person (a user). Typically, the initiation is effected by the user operating an electrical switch, which causes a controller to operate the device. Operation includes firstly injecting a needle into the user and then causing the injection of medicament into the patient's tissue. Installation of a bolus injector device is quite similar to installation of a patch pump, although patch pumps typically are used for basal delivery and remain installed for longer periods of time.

### SUMMARY

A first aspect of the invention provides an injection device comprising:
a housing;
a securing arrangement for securing the injection device against an injection site of a patient;
a needle insertion mechanism configured to cause insertion of a needle into a patient when the injection device is secured against the injection site of the patient;
a medicament delivery arrangement configured to cause injection of a medicament into tissue of the patient at the injection site; and
a medicament retention sensor configured to detect retention of medicament in the tissue of the patient at the injection site,

wherein the injection device is configured to respond to detection of swelling of the tissue of the patient at the injection site by causing provision of an alert and/or by causing suspension or halting of injection of medicament. This can improve user comfort and/or health in that, after tissue swelling is detected, further swelling can be avoided or minimized.

The injection device may be configured to respond to detection of swelling of the tissue of the patient at the injection site by causing provision of an alert without causing suspension or halting of injection of medicament. This can improve user comfort and/or health in that, after tissue swelling is detected, further swelling can be avoided or minimized by the patient or another user taking action to prevent further medicament delivery.

The injection device may be configured to respond to detection of swelling of the tissue of the patient at the injection site by causing suspension or halting of injection of medicament and causing provision of an alert. This can improve user comfort and/or health in that, after tissue swelling is detected, further swelling can be avoided or minimized by the automatic ceasing of medicament delivery whilst alerting the user so that they can take remedial action.

The injection device may comprise an alerting transducer and wherein the injection device may be configured to respond to detection of retention of medicament in the tissue of the patient at the injection site by causing the alerting transducer to provide an alert. This can avoid the need for an external alerting transducer to provide the alert.

The injection device may be configured to respond to detection of swelling of the tissue of the patient at the injection site by causing suspension or halting of injection of medicament without causing provision of an alert.

The securing arrangement may comprise an adhesive provided on an exterior surface of the housing.

The medicament retention sensor may comprise at least two electrical contacts located such as to contact the skin of the patient adjacent the injection site and may be configured to detect retention of medicament in the tissue of the patient at the injection site by detecting a change in an electrical property of an external path between the at least two electrical contacts.

The medicament retention sensor may comprise a camera configured to include in its field of view an area of the patient's skin around the injection site when the injection device is secured against the injection site of the patient.

The medicament retention sensor may be configured to monitor features of the patient's skin and determine when features are moving relatively apart from one another, thereby to detect retention of medicament.

The securing arrangement may comprise a belt or strap that extends around a limb of the patient and compresses the housing against the injection site of the patient.

The medicament retention sensor may be configured to detect a force in or extension of the strap or belt.

The medicament retention sensor may be configured to monitor measures of force in or extension of the strap or belt compared to a measure obtained at an earlier time.

The medicament retention sensor may be configured to eliminate or compensate for movements of the user. This can allow the medicament retention sensor to isolate increases in size of the limb of the patient resulting from medicament retention.

The device may be a bolus injector device.

The invention also provides a device as above with a container of medicament.

The invention also provides a method of controlling an injection device comprising: a housing, a securing arrangement for securing the injection device against an injection site of a patient, a needle insertion mechanism, a medicament delivery arrangement, and a medicament retention sensor, the method comprising:
using the medicament delivery arrangement to cause injection of a medicament into tissue of the patient at the injection site;
using the medicament retention sensor to detect retention of medicament in the tissue of the patient at the injection site; and
responding to detection of swelling of the tissue of the patient at the injection site by causing provision of an alert and/or by causing suspension or halting of injection of medicament.

### BRIEF DESCRIPTION OF THE DRAWINGS

Embodiments of the invention will now be described, by way of example only, which reference to the accompanying drawings in which;

FIG. 1 is a schematic cross-section of a bolus injector device according to various embodiments of the invention;

FIG. 2 is a schematic cross-section through the FIG. 1 bolus injection device including a strap or belt, according to embodiments of the invention;

FIG. 3 is a view of the bottom surface of the FIG. 1 bolus injection device according to further embodiments of the invention;

FIG. 4 is a schematic cross-section through an injection device according to still further embodiments of the invention; and

FIG. 5 is a flow-chart illustrating operation of the FIGS. 1 to 4 injection devices according to embodiments of the invention.

### DETAILED DESCRIPTION

Briefly, this specification discloses embodiments of an injection device, for instance a bolus injector device or a large volume device (LVD), for delivering bolus injections. The injection device is configured to be secured against the skin of a patient at an injection site. The injection device includes a needle insertion mechanism, that may be powered or may be manually operable, which causes insertion of a needle into a patient when the device is installed on the patient's skin. The needle can for instance be a hollow needle or a trocar of a cannular system, leaving a flexible tube in the patient for the injection phase.

The injection device includes a sensing arrangement configured to detect that medication is being retained in the tissue of the patient at the injection site. In response, the injection device is configured to provide an alert to the user and/or stop injecting the medicament into the patient. The condition that is detected is an edema condition. This is caused by an abnormal accumulation of liquid in the tissue, and not by the normal temporary presence of medicament as it enters and is absorbed into the tissue before being carried away from the injection site.

There are a number of possible forms for the sensor for detecting the tension of medicament in the tissue of the patient. This specification describes the injection device having electrical contacts that are in contact with the skin of the patient during use. By monitoring the resistance or conductivity of a path through the tissue of the user between the contacts, or the capacitance across the contacts, the retention of medicament can be detected. This specification also describes another option in which the device is secured to the patient by the use of strap, and the strain on or extension of the strap is monitored so as to detect the retention of medicament in the tissue of the patient. In a still further embodiment, a camera is used to monitor the skin of the user in the region around the injection site. Images from the camera are processed to detect a change in the apparent distance between features on the user's skin, thereby inferring swelling of the tissue around the injection site.

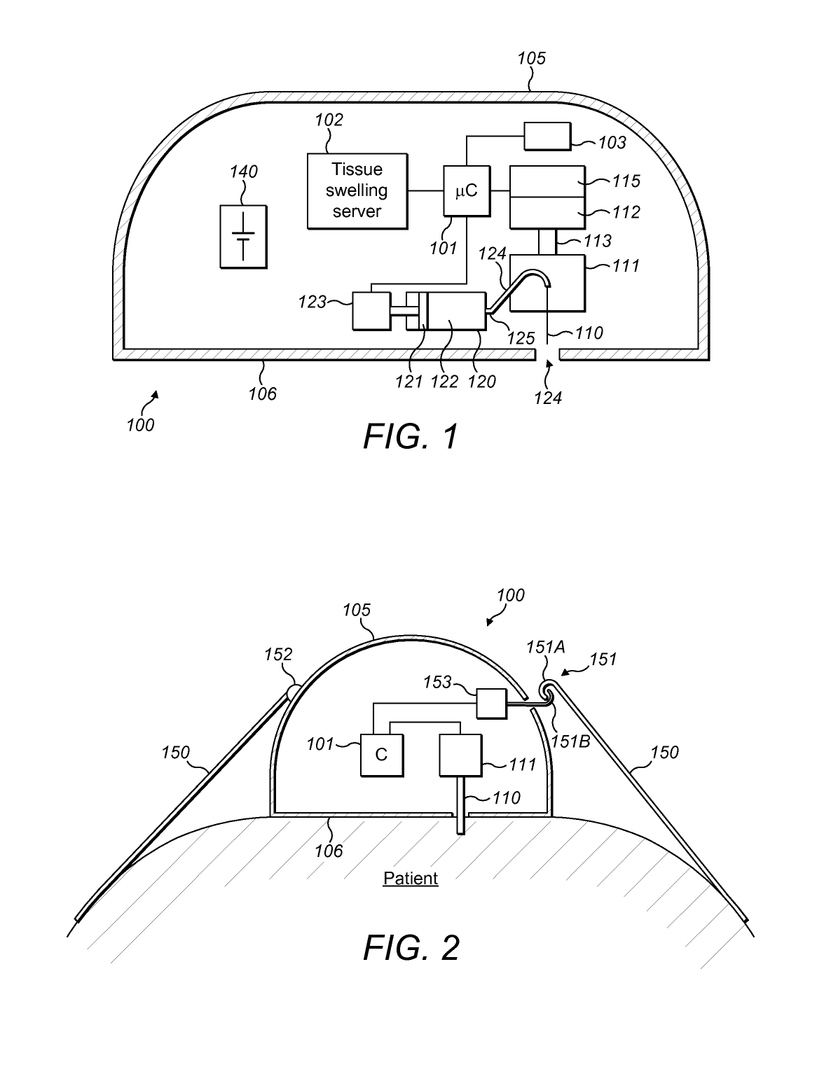

Referring firstly to FIG. 1, a bolus injector device according to various embodiments of the invention is shown in schematic form. The bolus injector device 100 includes numerous components, key ones of which will now be described. In the following, the user of the device is assumed to be the patient intended to receive medicament, although the user may be a different person to the patient.

The bolus injector device 100 includes a controller 101, that is configured to control operation of various components, as will be apparent from the below description.

The bolus injector device 100 includes a housing 105. The shape of the housing 105 may take any suitable form. Here, the housing 105 is shown to a have a cross-section that is substantially dome shaped. The housing 105 includes a lower surface 106, which is substantially planar and which is configured to be placed on the skin of a user during operation. The lower surface 106 may be provided with a layer of adhesive 133, so as to allow the bolus injector device 100 to be secured to the user's skin during medicament delivery. The housing 105 also includes an upper surface, which is curved in this example. The housing 105 defines an interior cavity in which most of the components of the bolus injector device 100 are located.

The bolus injector device 100 includes a tissue swelling sensor, operation of which is described in detail below.

The bolus injector device 100 includes an output transducer 103. This may be operable to produce an alert sound, a visible alert, or both.

The bolus injector device 100 includes a needle 110 and a needle insertion mechanism 111. The needle insertion mechanism 111 is controllable by the controller 101 to cause the needle 110 to extend through a needle aperture 114 in the housing 105 such as to pierce the skin of a user on whom the patch device is located for medicament delivery. In FIG. 1, the needle 110 is shown in a retracted position, in which a tip of the needle 110 does not extend through the needle aperture 114. After the needle insertion mechanism 111 has operated, the needle 110 extends through the needle aperture 114. The tip of the needle 110 may for instance be inserted by distance of 5 to 10 mm through the needle aperture 114 so as to be inserted into tissue of a user to the same depth. The needle in this embodiment is a hollow needle having a bore.

The needle 110 is driven by the needle insertion mechanism 111 to be inserted into the user by a needle insertion mechanism drive 112. The needle insertion mechanism driver 112 may for instance be an electric motor or a spring release mechanism. Energy for driving the needle insertion mechanism driver 112 comes from a needle driving energy source 115. The form of the needle driving energy source 115 corresponds to the form of the needle insertion mechanism driver 112, and is discussed below. A connecting mechanism 113 connects the needle insertion mechanism driver 112 to the needle insertion mechanism 111. The connecting mechanism 113 provides mechanical coupling between these two components. The needle insertion mechanism driver 112 and the needle driving energy source 115 are controlled by the controller 101.

In some embodiments the needle insertion mechanism 111 is manually operated and is not powered from within the bolus injector device 100. In these embodiments, the needle energy driving source 115 and the needle insertion mechanism driver 112 are omitted. The needle insertion mechanism driver 112 is substituted with a mechanism for communicating user-applied work to movement of the needle 110 via the needle insertion mechanism 111. The communicating mechanism may translate user-provided work in the form of a rotation movement or a slide movement or a depression movement into movement of the needle 110 to be inserted into the tissue of the patient.

A medicament cartridge 120 is provided in the housing 105 of the bolus injector device 100. The medicament cartridge 120 may for instance include a vial formed of glass. A plunger 121 is provided within the cartridge 120 at an opposite end to a medicament delivery aperture 125. Between the plunger 121 and the end of the medicament cartridge 120 that includes the medicament delivery aperture 125 is defined a volume that is filled with medicament 122.

A medicament expelling driver 123 is mechanically coupled to the plunger 121. The medicament expelling driver 123 is controllable by the controller 101 to move the plunger 121 along the medicament cartridge 120. When so controlled, the force provided by the plunger 121 on the medicament 122 causes it to be expelled through the medicament delivery aperture 125 and along a medicament delivery tube 124 to the needle 110, in particular, the end of the needle 110 that is opposite to the end that is inserted into the user. When so operated, the medicament 122 is caused to be expelled through the bore of the needle 110. The expelling of the medicament in this way can be described as pumping. The flow rate of medicament expulsion can be set to some extent by configuration of the bolus injector device 100, but it is dependent also on physical characteristics of the patient's tissue at the injection site.

An electrical power source in the form of a battery 140 is provided. The battery 140 provides electrical power to the controller 101. It may also provide electrical power the plunger driver 123, if this is an electrically driven device. The battery 140 may also constitute the needle driving energy source 115, that is to say the needle driving energy source 115 and the battery 140 may be combined into a single component.

It will be appreciated that, as medicament 122 is injected into the patient through the needle 110, the medicament is absorbed into the tissue of the patient. If the site of the injection is correct and the depth of the injection of the needle into the patient is correct, the medicament 122 is relatively quickly absorbed by the tissue of the patient and is carried away from the injection site, and is dispersed around the body of the patient. If, however, there is a problem, the medicament can be retained locally to the injection site and not be carried away sufficiently quickly. In such a situation, the tissue of the patient at and around the injection site will swell as medicament is injected. Such swelling can be uncomfortable or even painful to the user, and in extreme cases might cause tissue damage and possibly present other undesirable consequences.

Referring to FIG. 2, a bolus injector device 100 according to various embodiments is shown secured to a patient by a strap or belt 150. The strap may take any suitable form, and may for instance be webbing, ribbon etc. The strap may be made of any suitable material.

The strap 150 is secured at a first end to a first strap securing arrangement 152 that is provided on an exterior surface of the housing 105 of the bolus injector device 100. At the other end, the strap 150 is secured to a different part of the exterior surface of the housing 105 of the bolus injector device 100. In particular, a second securing arrangement 151 is provided. In this example, the first and second securing arrangements 151, 152 are separated from one another on the surface of the bolus injector device 100, although they may instead be co-located.

The second securing arrangement 151 includes a first component 151A that is affixedly connected to or forms part of the strap 150. It also includes a second component 151B that is connected to or forms part of the main part of the bolus injector device 100. The first second parts 151A and 151B of the second securing arrangement 151 are configured so as to be able to mate in a releasable manner. In this way, the second end of the strap 150 can be connected releasably to the housing 105 of the bolus injector device 100.

The strap 150 extends around a limb, for instance an arm or a leg, of the patient. The strap 150 is in tension, which causes the housing 105 of the bolus injector device 100 to be secured against the patient. To install the bolus injector device 100 on the patient, the patient, or another user, places the housing 105 at the desired injection site. The first end of the strap 150 is secured to the housing 105 by the first securing arrangement 152. The user then takes the second of the strap 150 and connects it to the housing 105 of the bolus injector device 100 by mating the two parts 151A and 151B of the second securing arrangement 151. After the second securing arrangement 151 has been so assembled, the strap 150 secures the housing 105 to the limb of the patient. In this position, the lower surface 106 of the housing 105 is supported against the skin of the patient at the injection site. As can be seen from the Figure, this causes the tissue of the patient to distort such that it contacts a large proportion or all of the lower surface 106 of the housing.

The housing 105 of the bolus injector device 100 houses a sensing arrangement 153, which is connected to the second securing arrangement 151, in particular the second component 151B thereof. As such the sensing arrangement 153 is operable to detect forces in and/or extension of the strap 150. The sensing arrangement 153 forms part of the tissue swelling sensor 102.

In some implementations, the strap 150 is elastic and thus can be stretched so that its length varies upon application of a force pulling the first and second ends of the strap away from each other. Here, the sensing arrangement 153 comprises a force sensor such as a strain gauge. As such, the sensing arrangement 153 is operable to detect a force experienced along the length of the strap 150, or at least force that is provided by the second end of the strap 150.

In other implementations the strap is substantially inelastic, so its length cannot change or cannot change significantly upon application of a stretching force. Here, the sensing arrangement 153 includes a mechanism by which a mechanical connector between the sensing arrangement 153 and the second part 151B of the securing arrangement 151 can be varied in length. For instance, the sensing arrangement 153 may comprise a coiled cord, cable, wire or strap. Here, a force provided in a direction away from the housing 105 of the bolus injector device 100 by the second end of the strap 150 causes the cord, wire, cable or strap to be unwound. The sensing arrangement 153 in these embodiments is operable to detect an extent to which the wire, cable, cord or strap is extended. This may sense directly the amount of cord, wire cable or strap, or it may detect an amount of rotation of a drum or bobbin on which the cord, wire, cable or strap is wound.

However it is configured, the sensing arrangement 153 is operable to detect a force that is provided on the strap 150 by the limb of the patient around which the strap 150 is extended.

The controller 101 is configured to monitor the signals provided by the sensing arrangement 153 and to detect from the signals whether the tissue of the patient is retaining medicament to an unsatisfactory degree. By monitoring force in or extension of the strap 150, the controller 101 and the sensing arrangement 153 are able to detect when the tissue of the patient adjacently in the injection site is swollen by the retention of medicament. Swelling is inferred by detecting an increase in force in or extension of the strap 150.

An increase in force in or extension of the strap 150 may be detected by comparing the output of the sensor 153 to a threshold. The threshold is set by the force/extension prior to the injection starting or soon after the beginning of the injection. The threshold may be set relative to the average force/extension over a period of time prior to the injection starting or at the beginning of the injection. The threshold may be set as a certain amount, which may be fixed or may be a proportion, above the force/extension prior to or at the beginning the injection.

Advantageously, the increase in force in or extension of the strap 150 is detected by low pass filtering the output of the sensor 153 and comparing the filtered sensor output to a threshold. The threshold may be set as outlined above. Low pass filtering of the output of the sensor 153 prevents an incorrect inference of tissue swelling caused by the tensing of muscles in, or other movement of, the limb to which the bolus injection device is attached. The time constant of the low pass filter may for instance be of the order of a few second or a few tens of seconds. Alternatively, the increase in force in or extension of the strap 150 caused by movement or muscle contraction etc. may be filtered out of compensated in some other way. Without being able to filter out or compensate patient movement and/or muscle contraction, the patient may need to remain relatively motionless, at least as regards the limb on which the bolus injector device 100 is installed, in order to ensure reliable sensing of tissue swelling by the sensor 102.

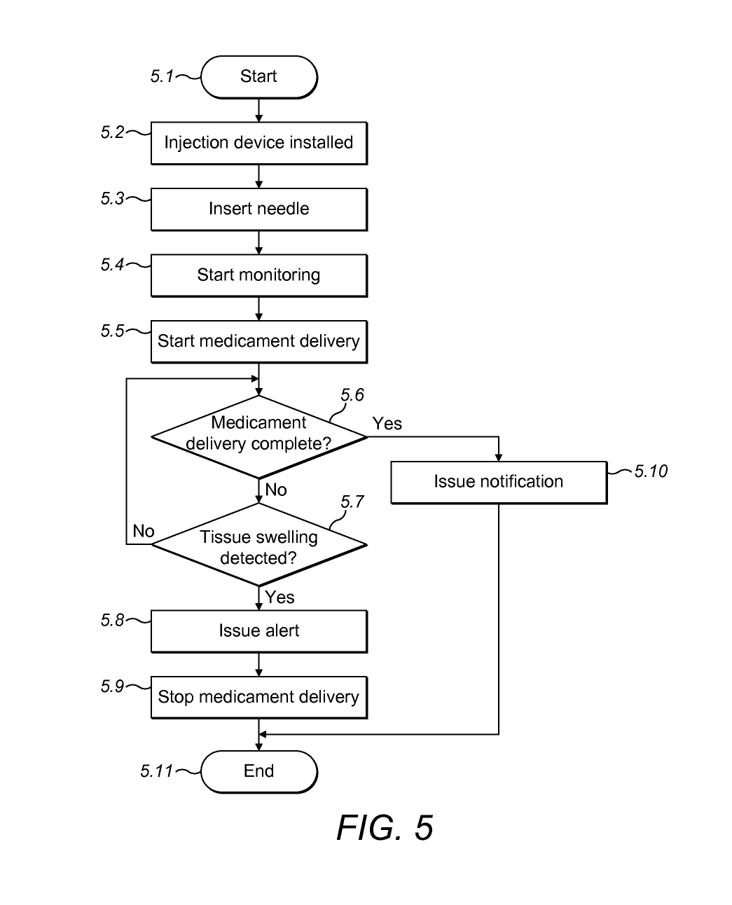

In response to detecting swelling, the bolus injector device 100 provides an alert to the user, notifying them that there is a potential problem. The bolus injector device 100 alternatively or additionally ceases or suspends (i.e. pauses) the injection of medicament through the needle 110 into the user. This can occur for instance by the controller 101 controlling the medicament expulsion driver 123 to cease delivering medicament 122. This is explained in more detail below with reference to FIG. 5.

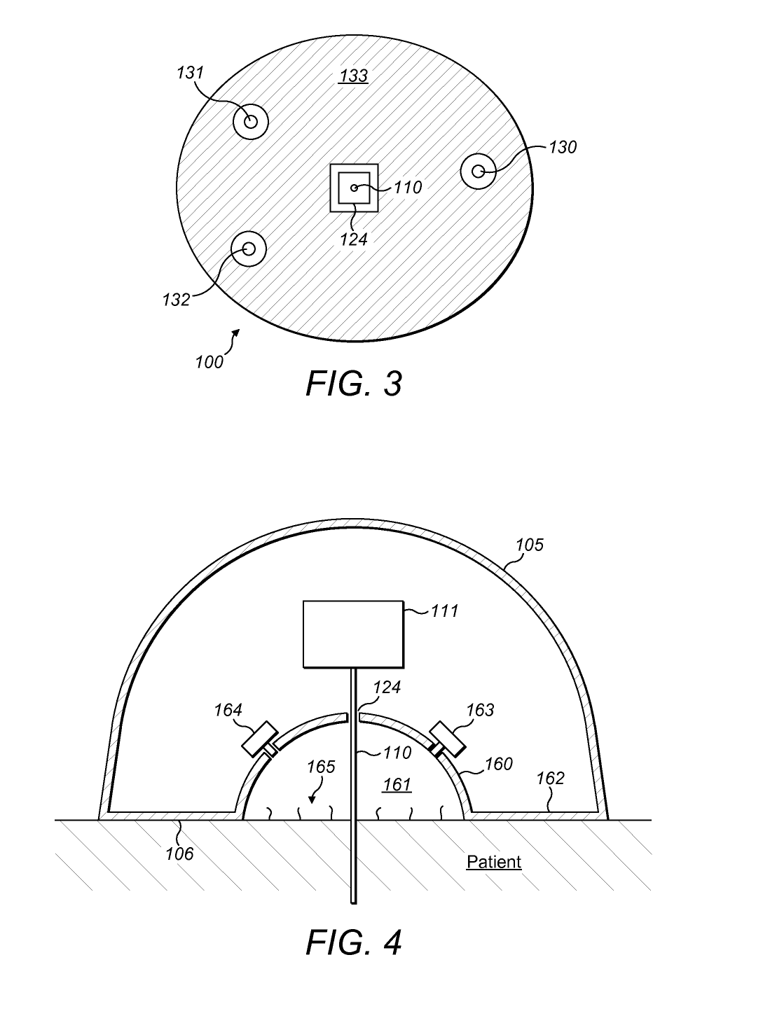

Referring now to FIG. 3, other embodiments are shown. Here, the bottom surface 106 of the bolus injector device 100 is shown. The needle aperture 124 in shown in the middle of the device, and the needle 110 is visible therethrough.

First and second conductive contacts 130, 131 are provided on the underside surface 106. The first and second contacts 130, 131 are separated from one another. In this example, each is near the edge of the device and they are on opposite sides of the needle aperture 124. As such, there is a significant distance between the first and second contacts 130, 131 and this example. The first and second contacts 130, 131 form part of the tissue swelling sensor 102. The tissue swelling sensor 102 also includes a driver circuit (not shown) that is configured to provide an electrical signal across the first and second contacts 130, 131 and to measure a resulting electrical parameter.

The electrical signal is for instance a direct current (DC) voltage. The parameter for instance is the current that flows in the resulting circuit. As such, the tissue swelling sensor 102 of this embodiment is operable to measure the resistance (or conductivity, which is the converse parameter) of the patient's skin near the injection site. In particular, when the bolus injector device 100 is secured against the skin of the user at an injection site, the contacts 130, 131 both contact the skin of the patient. By operation of the tissue swelling sensor 102, the resistance (or conductivity) of the patient, or in particular the part of the patient that is present in the vicinity of the first and second contacts 130, 131, can be measured. A retention of medicament in the tissue of the patient at the injection site results in a decreased resistance of the tissue, which can thus be detected by the tissue swelling sensor 102 in order ascertain that medicament retention is occurring.

The electrical signal alternatively may be an alternating current (AC) voltage. The parameter here may be the current that flows in the resulting circuit, and a measure of resistance (or conductivity) of the patient's skin near the injection site is used to detect retention of medicament in the tissue of the patient at the injection site. Alternatively the parameter is capacitance, and a change in capacitance is used to detect retention of medicament in the tissue of the patient at the injection site. The change in the parameter is detected between a current time and a time after the bolus injector device 100 was installed on the patient and before or soon after the start of medicament delivery. The parameter is detected relatively frequently, for instance every 5, 10, 20 or 30 seconds.

Optionally a third contact 132 is provided on the lowermost surface 106 of the bolus injector device 100. The use of a third contact 132 allows a number of different resistance/conductance/capacitance measurements to be made.

Resistance/conductance/capacitance measurements can be made between any two of the three contacts 130 to 132. The use of three contacts also allows the tissue swelling sensor 102 to detect the resistance/conductance/capacitance of the patient's tissue in the event that one of the contacts 130 to 132 should not have a proper electrical connection with the users skin, for instance because of a barrier in between the skin and the contact.

The lowermost surface 106 of the bolus injector device 100 is provided with an adhesive layer 133. The adhesive layer 133 extends to the edge or almost the edge of the bolus injector device 100. Apertures in the adhesive layer are formed around the contacts 130-132. Also, an aperture in the adhesive layer is formed around the needle aperture 124. Alternatively, the adhesive layer 133 may include only apertures that are the same size and shape as the contacts 130 to 132 and/or the needle aperture 124.

The contacts 130 to 132 may take any suitable form. They are formed of a conductive material, for instance a metal such as aluminum or copper or an alloy thereof. They may protrude from the general plane of the lower surface 106 of the bolus injector device 100. For instance, they may be domed in shape so as to protrude from the surface 106. Having the contacts 130 to 132 as protrusions from the surface allows them to form a better mechanical connection with the patient's skin than might otherwise be achieved. Providing the contacts 130-132 with a curved surface, such as a domed profile, minimizes the possibility of discomfort or injury to the patient during installation or use of the bolus injector device 100.

An electrically conductive material, for instance an electrically conductive gel, may be provided on the surfaces of the contacts 130-132. This can assist in providing a good electrical connection between the patient and the tissue swelling sensor 102. In some embodiments, the electrically conductive gel is protected before the bolus injector device 100 is used by a backing layer that extends over the whole of the lowermost surface 106 of the bolus injector device 100. This backing layer may serve as dual purpose backing for the adhesive layer 133 and the electrically conductive gel layers on the contacts 130 to 132.

Some other embodiments will now be described with reference to FIG. 4.

In FIG. 4, the housing 105 of the bolus injector device 100 includes a central cavity 161 that is formed by a cavity defining part 160 of the housing 105. The cavity defining part 160 extends around the needle 110, and thus extends around the injection site. The needle aperture 124 is formed at the central part of the cavity defining part 160 of the housing 105.

The cavity defining part 160 is configured such as to provide the cavity 161 with appropriate dimensions. For instance, an opening at the lowermost part of the cavity 161, at which the patient's skin is found when the bolus injector device 100 is installed on the patient, may have a diameter of between 0.5 and 2 cm, advantageously around 1 cm. The opening of the cavity 161 may be circular, square or take any other shape. In the embodiment illustrated in FIG. 4, the cavity defining part 160 is dome shaped, so as to provide a hemispherical cavity 161, but it may be of any other suitable shape. An effect of the provision of the cavity 161 is that an area of skin near the injection site is constrained at its periphery (by the adhesive 133 on the lowermost surface 106) but not constrained by the bolus injector device 100 at the center. The cavity also allows that unconstrained part of the skin to be monitored.

An illumination source 164 is provided within the bolus injector device 100 so as to illuminate the patient's skin in the area surrounding the injection site. The illumination source 164 may take any suitable form, and may for instance be one or more light emitting diodes. The illumination source may be substantially monochrome or it may have a wide spectrum, or it may take any form in between these two extremes.

A camera 163 is also provided on the bolus injector device 100. The camera has a field of view that includes at least part of the patient's skin that is illuminated by the illumination source 164. The camera 163 is operable to detect light in at least one wavelength that is omitted by the source of illumination 164. The camera 163 forms part of the tissue swelling sensor 102.

The tissue swelling sensor 102 is configured to capture images of the scene before the camera 163 at intervals and to process the images. The tissue swelling sensor 102 is configured to ensure that the source of illumination 164 is controlled to illuminate the scene before the camera at the time that the images are captured for processing. The source of illumination 164 may be controlled to emit light continually, or it may be controlled to emit light only at times when the camera 163 is controlled to capture images.

The tissue swelling sensor 102 is configured to process images captured by the camera 163 to identify features of the patient's skin. The tissue swelling sensor 102 is configured also to detect relative movements of these features. The features may for instance be pores, moles, freckles, hairs etc. Any feature that is present on the skin of the user and which is visible may be suitable for monitoring.

The tissue swelling sensor 102 is configured to compare images taken at different times and determine from the images whether features on the patient's skin that are present in the images are moving apart. If they are moving apart, that is the distance between them is increasing, this is indicative of swelling of the tissue of the patient around the injection site, and is used by the tissue swelling sensor 102 to identify that there is retention of medicament in the patient's tissue around the injection site.

Detection of increasing separation of features on the patient's skin between two images can be made in any suitable way. It may be detected for instance by counting the number of pixels that separate two features. It may be achieved alternatively by comparing two images from different times and calculating the difference therebetween, and using the difference to detect that features are moving apart. The detection of movement of features advantageously is made between a latest captured image and an image captured before the start of medicament delivery or hotly thereafter.

Operation of the above-described bolus injector device 100 will now be described with reference to FIG. 5.

The operation starts at step 5.1. At step 5.2, the bolus injector device 100 is installed on the patient at the injection site. In the case of the bolus injector device 100 including an adhesive means 133 for securing, this involves the user removing a backing layer to reveal the adhesive layer present on the lowermost surface 106 of the bolus injector device 100 and then placing the device against their skin at the injection site. In the case of the injection device including or being usable with the strap or belt 150, this involves the user securing the bolus injector device 100 at the injection site by connecting the two parts 151A and 151B of the second securing means 151. The device 100 may be configured to detect that the device is properly installed on the patient's skin, or the installation of the device may be inferred by the user initiating operation of the device 100, for instance by operating a ‘start’ switch.

At step 5.3, the needle 110 is inserted into the injection site of the patient. In embodiments where the needle insertion mechanism is powered by the needle insertion mechanism driver 112, this involves the controller 101 powering the needle insertion mechanism 111. In particular, the controller 101 controls the needle driving energy source 115 and the needle insertion mechanism driver 112 to provide a driving force to the needle insertion mechanism 111 such that the needle 110 is begun to be inserted into the patient's tissue through the needle aperture 114. This continues until the bolus injector device 100, in particular the controller 101, detects that the needle insertion is complete. This may occur for instance using feedback from the needle insertion mechanism driver 112, or using feedback from the needle insertion mechanism 111 or through a separate sensor (not shown). In the case of a manually operable needle insertion mechanism 111, this involves the patient or another user operating the mechanism 111 to insert the needle 110 into the injection site of the patient. In either case, operation of the bolus injector device 100 is started at around about this time by action of the patient or another user.

At step 5.4, the tissue swelling sensor 102 starts monitoring the tissue of the patient around the injection site. In the case of the FIG. 2 embodiments involving the strap 150, this involves starting to monitor the force present in the strap 150, by way of the sensor 153. In the case of the FIG. 3 embodiments, this involves the tissue swelling sensor 102 detecting a measure of the resistance, conductivity or capacitance of the patient's tissue between two of the contacts 130-132, or between some other combination of the contacts 130-132. It may involve the tissue swelling sensor 102 determining an average measurement over a period of time of for instance a few seconds. In the case of the FIG. 4 embodiments, step 5.4 involves the tissue swelling sensor 102 controlling the camera 163 to capture one or more images, by which the locations of features on the patient's skin before medicament delivery starts can be ascertained.

Steps 5.4 and 5.3 may be performed in the reverse order, but having the needle 110 inserted into the patient before monitoring starts is preferred.

Following step 5.4, medicament delivery is started at step 5.5. The medicament delivery may commence immediately in response to the detecting that the needle insertion is complete or it may require another trigger condition. For instance, it may be dependent on a user operating an electrical switch or some other input device on the bolus injector device 100. The trigger may alternatively be a timer expiring, where a timer is started when the needle insertion is detected to be complete at step 5.4. For instance, the medicament delivery may start 5 or 10 seconds after the needle insertion is detected to be complete. The step 5.5 of starting the medicament delivery comprises the controller 101 controlling the medicament expulsion driver 123 to supply force on the plunger 121, which then causes expulsion of the medicament 122 from the medicament cartridge 120 through the medicament delivery aperture 125 and along the medicaments delivery tube 124 and through the bore of the needle 110 into the tissue of the user.

At step 5.6, the controller 101 of the bolus injector device 100 detects whether the medicament delivery is complete. This may occur in any the suitable way. For instance, it may involve detection based on feedback provided by the medicament expulsion driver 123 that the plunger driver has stopped, for instance because it has reached the full extent of travel of the plunger 121. It may alternatively be detected in any other way, for instance in response to detecting that an integral of flow rate signals provided by a flow meter (not shown) exceeds a threshold, indicating that a desired medicament dose has been delivered through the needle 110.

In response to the bolus injector device detecting at step 5.6 that the medicament delivery is not yet complete, at step 5.7 the bolus injector device 100 determines whether tissue swelling is detected. The nature of this step depends on the type of tissue swelling sensor 102 that is used in the bolus injector device 100, and some alternatives are described above.

In response to the bolus injector device detecting at step 5.7 that there no tissue swelling is detected, or that this condition is not detected with the requisite level of likelihood, the operation returns to determine again whether medicament delivery is complete at step 5.6.

In response to the bolus injector device detecting at step 5.7 that tissue swelling is detected, particularly that this condition is detected with the requisite level of likelihood, the operation proceeds to step 5.8. Here, the bolus injector device 100 issues an alert. This can occur in any suitable way, for instance through operating the alerting transducer 103 to provide a notification signal such as a flashing light or a light of a certain color, or a certain sound.

Then, at step 5.9, the bolus injection device ceases medicament delivery. This is achieved by the controller 101 causing the medicament expulsion driver 123 to cease moving the plunger 121. The ceasing of medicament delivery at step 5.9 may be permanent or temporary. It may involve the bolus injector device 100 operating such that it is unable (through its software programming or its mechanical configuration) to deliver any further medicament. It may alternatively resume medicament delivery in response to a detection that the tissue swelling condition is no longer present. It may further alternatively resume medicament delivery in response to a detection of a user input indicating that medicament delivery resumption is required or that the tissue swelling condition is no longer present. In the interim period, the patient may have relocated the bolus injector device 100 to a different injection site.

In some embodiments, the bolus injector device 100 issues the alert at step 5.8 but does not stop medicament delivery, or does not stop medicament delivery until receiving a user input indicating that medicament delivery is to be stopped.

In other embodiments, the bolus injector device 100 stops or pauses medicament delivery but does not issue an alert. In these embodiments, the bolus injector device 100 advantageously is configured to resume medicament delivery when the tissue swelling condition is detected no longer to be present. The use of a sensitive tissue swelling sensor 102 may be particularly advantageous in these embodiments to ensure that patient discomfort is avoided or kept to a minimum. In these embodiments, it may be that medicament delivery is controlled such that an extent of tissue swelling resulting from delivery of the medicament is kept below a threshold amount. This can help to deliver a quantity of medicament as quickly as possible whilst not causing an undesired level of tissue swelling. This can be particularly useful in large volume medicament delivery.

In response to the bolus injector device detecting at step 5.6 that the medicament delivery is complete, at step 5.10 the bolus injector device 100 notifies the user that the medicament delivery is complete. This can occur in any suitable way, for instance through operating the alerting transducer 103 to provide a notification signal such as a flashing light or a light of a certain color, or a certain sound. The notification that that medicament delivery is complete is different to the alert provided at step 5.8, so that the user can distinguish between a medicament delivery complete event and a tissue swelling event.

After step 5.9 or step 5.10, the operation ends at step 5.11.

The term “drug” or “medicament”, as used herein, means a pharmaceutical formulation containing at least one pharmaceutically active compound. In some embodiments, the pharmaceutically active compound can have a molecular weight up to 1500 Da or may include a peptide, a protein, a polysaccharide, a vaccine, a DNA molecule, an RNA molecule, an enzyme, an antibody or a fragment thereof, a hormone or an oligonucleotide, or a mixture of the above-mentioned pharmaceutically active compound. Various types or subtypes of compounds are also contemplated. For example, RNA may include RNAi, siRNA, or miRNA. In other embodiments, the pharmaceutically active compound can be useful for the treatment or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis or rheumatoid arthritis. In some embodiments, the pharmaceutically active compound can comprise at least one peptide for the treatment or prophylaxis of diabetes mellitus or complications associated with diabetes mellitus such as diabetic retinopathy. The pharmaceutically active compound can also comprise at least one human insulin or a human insulin analogue or derivative, glucagon-like peptide (GLP-1) or an analogue or derivative thereof, or exendin-3 or exendin-4 or an analogue or derivative of exendin-3 or exendin-4 or a pharmaceutically acceptable salt or solvate thereof.

Insulin analogues can include, for example, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives can include, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-'decanoyl) human insulin.

Exendin-4 can include, for example, Exendin-4(1-39).

Hormones can include, for example, hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, or Goserelin.

A polysaccharide can include, for example, a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies can include generally globular plasma proteins (˜150 kDa) that are also known as immunoglobulins which share a basic structure. As they can have sugar chains added to amino acid residues, they may also be classified as glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that can include four polypeptide chains; two heavy chains and two light chains connected by disulfide bonds between cysteine residues. Each heavy chain can be about 440 amino acids long; each light chain can be about 220 amino acids long. Heavy and light chains may each contain intra-chain disulfide bonds which stabilize their folding. Each chain is composed of structural domains called Ig domains. These domains typically contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains y, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of antibodies can be similar, the unique property of a given antibody is determined by the variable (V) regions, as detailed above. More specifically, variable loops, often three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is usually the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their inter-chain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H inter-chain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion. Pharmaceutically acceptable solvates are for example hydrates.

In some embodiments, medicaments of various viscosities can be injected. For example, viscosity could range from about 3 to about 50 cP. In other embodiments, viscosity could be less than about 3 cP or greater than about 50 cP. Injection can further include delivering a medicament to a sub-cutaneous, an intra-muscular, or a transdermal location within a patient's body. The medicament can be in the form of a liquid, gel, slurry, suspension, particle, powder, or other type.

Typical injection volumes can range from about 1 mL to about 10 mL. Rates of injection may be about 0.5 mL/min, about 0.2 mL/min, or about 0.1 mL/min. Such injection profiles may be generally constant in flow rate, generally continuous in duration, or both generally constant and generally continuous. These injections can also occur in a single step of administration. Such injection profiles may be referred to as bolus injections.

Delivery devices functioning with such medicaments may utilize a needle, cannula, or other injection element configured to deliver a medicament to the patient. Such an injection element may, for example, have an external size or diameter of 27 G or less. Further, the injection element could be rigid, flexible, and formed using a range of one or more materials. And in some embodiments, the injection element may include two or more components. For example, a rigid trocar may operate in conjunction with a flexible cannula. Initially, both the trocar and cannula may move together to pierce the skin. The trocar may then retract while the cannula remains at least partially within the target tissue. Later, the cannula may separately retract into the delivery device.

It will be appreciated that the above embodiments are not limiting on the scope of the invention. Various alternatives will be envisaged by the skilled person and are intended to be within the scope of the invention. Some such alternatives will now be described.

Instead of the alerting transducer being part of the bolus injector device 100, it may instead be external. For instance it may form part of a mobile phone or other device. To provide the alert, the bolus injector device 100 communicates, preferably wirelessly, with the other device with an instruction to provide an alert.

In the above, the injection device 100 is a bolus injector pump device that is configured to inject a bolus dose over a period of some minutes or hours. The injection device may instead be another type of infusion pump or some other form of injection device. The embodiments of the invention are particularly suited to bolus injections, but the injection device may instead be of the basal type.

The needle insertion mechanism driver 112 may take any suitable form. It may for instance include an electric motor and a gear mechanism that causes insertion of the needle 110 into the user. It may alternatively be a mechanical spring based mechanism. In this case the needle driving energy source 115 is a preloaded spring, and the needle insertion mechanism driver 112 is a spring release mechanism that causes force from the spring to be communicated to the needle insertion mechanism 111 thereby to insert the need 110 into the user.

Alternatively, the needle insertion mechanism driver 112 may be a gas or fluid pressure operated mechanism, in which case the needle driving energy source 115 is either a reservoir of pressurized gas or a chemical system in which two or more chemicals are mixed together to produce gas or fluid pressure.

1. An injection device comprising:
a housing;
a securing mechanism configured to secure the injection device against an injection site of a patient;
a needle insertion mechanism configured to cause insertion of a needle into a patient when the injection device is secured against the injection site of the patient;
a medicament delivery mechanism configured to cause injection of a medicament into tissue of the patient at the injection site; and
a medicament retention sensor configured to detect retention of medicament in the tissue of the patient at the injection site,
wherein the injection device is configured to respond to detection by the medicament retention sensor of medicament in the tissue of the patient at the injection site by at least one of causing provision of an alert and suspending or halting the injection of the medicament.

2. A device as claimed in claim 1, wherein the injection device is configured to respond to the detection of medicament in the tissue of the patient at the injection site by causing provision of an alert without suspending or halting injection of medicament.

3. A device as claimed in claim 1, wherein the injection device is configured to respond to the detection of medicament in the tissue of the patient at the injection site by suspending or halting injection of the medicament and causing provision of an alert.

4. A device as claimed in claim 1, wherein the injection device comprises an alerting transducer and wherein the injection device is configured to respond to the detection of retention of medicament in the tissue of the patient at the injection site by causing the alerting transducer to provide an alert.

5. A device as claimed in claim 1, wherein the injection device is configured to respond to the detection of medicament in the tissue of the patient at the injection site by causing suspension or halting of injection of medicament without causing provision of an alert.

6. A device as claimed in claim 1, wherein the securing mechanism comprises an adhesive provided on an exterior surface of the housing.

7. A device as claimed in claim 1, wherein the medicament retention sensor comprises at least two electrical contacts configured to contact the skin of the patient adjacent the injection site and the medicament retention sensor is configured to detect retention of medicament in the tissue of the patient at the injection site by detecting a change in an electrical property of an external path between the at least two electrical contacts.

8. A device as claimed in claim 7, wherein the medicament retention sensor comprises a camera configured to include in a field of view of the camera an area of the patient's skin around the injection site when the injection device is secured against the injection site of the patient.

9. A device as claimed in claim 8, wherein the medicament retention sensor is configured to monitor features of the patient's skin and determine when features are moving relatively apart from one another and thereby detect retention of the medicament.

10. A device as claimed in claim 1, wherein the securing mechanism comprises a belt or strap configured to extend around a limb of the patient and compress the housing against the injection site of the patient.

11. A device as claimed in claim 10, wherein the medicament retention sensor is configured to detect a force in or extension of the strap or belt.

12. A device as claimed in claim 11, wherein the medicament retention sensor is configured to monitor measures of force in or extension of the strap or belt compared to a measure obtained at an earlier time.

13. A device as claimed in claim 11, wherein the medicament retention sensor is configured to eliminate or compensate for movements of the user.

14. A device as claimed in claim 1, wherein the device is a bolus injector device.

15. A device as claimed in claim 1, with a container of medicament.

16. The drug delivery device of claim 15, wherein the medicament comprises a pharmaceutically active compound.

17. A method comprising:
securing an injection device against an injection site of a patient, the injection device comprising a medicament retention sensor, a needle insertion mechanism, and medicament delivery mechanism;
causing the needle insertion mechanism to insert a needle into a patient when the injection device is secured against the injection site of the patient; and
causing the medicament delivery mechanism to inject a medicament into tissue of the patient at the injection site, the medicament retention sensor detecting retention of medicament in the tissue of the patient at the injection site and responding to a detection by causing one or more of: provisioning an alert, suspending the injection of the medicament, and halting the injection of the medicament.
